(19) United States
(12) Patent Application Publication
(10) Pub. No.: US 2019/0103189 A1
(43) Pub. Date: Apr. 4, 2019

(54) AUGMENTING EHEALTH INTERVENTIONS WITH LEARNING AND ADAPTATION CAPABILITIES

(71) Applicant: KONINKLIJKE PHILIPS N.V., Eindhoven (NL)

(72) Inventors: DANIEL JASON SCHULMAN, JAMAICA PLAIN, MA (US); JOYCA PETRA WILMA LACROIX, EINDHOVEN (NL); ARLETTE VAN WISSEN, CULEMBORG (NL); ANNERIEKE HEUVELINK-MARCK, EINDHOVEN (NL); DIETWIG JOS CLEMENT LOWET, EINDHOVEN (NL); CLIFF JOHANNES ROBERT HUBERTINA LASCHET, GULPEN (NL); JAN TATOUSEK, EINDHOVEN (NL); JAN VAN SWEEVELT, EINDHOVEN (NL)

(21) Appl. No.: 16/142,661

(22) Filed: Sep. 26, 2018

Related U.S. Application Data
(60) Provisional application No. 62/565,381, filed on Sep. 29, 2017.

(51) Int. Cl. G16H 50/30; G06N 99/00; G06Q 30/02

(52) U.S. Cl. CPC G16H 50/30; H04L 67/10; G06Q 30/0207; G06N 99/005

(57) ABSTRACT

In an embodiment, a computer-implemented method, comprising: receiving contextual information for a user; updating a user state based on the received contextual information; providing electronic interventions to the user over a first interval by executing a first intervention algorithm based on the updated user state; and providing electronic interventions to the user over a second interval based on executing a second intervention algorithm that maximizes a reward function based on a further updated user state of the user and the electronic interventions of the first interval, the second intervention algorithm of a different type than the first intervention algorithm.

### CROSS-REFERENCE TO RELATED APPLICATIONS

This patent application claims the priority benefit under 35 U.S.C. § 119(e) of U.S. Provisional Application No. 62/565,381 filed on Sep. 29, 2017, the contents of which are herein incorporated by reference.

### FIELD OF THE INVENTION

The present invention is generally related to electronic health (eHealth) applications.

### BACKGROUND OF THE INVENTION

Today, there are numerous eHealth applications (also referred to as behavior change support technologies), including healthy lifestyle promotion (e.g. physical activity, healthy diet, smoking cessation, etc.), treatment adherence promotion, and self-monitoring and self-management of chronic conditions. Extensive scientific studies suggest that user-tailoring/adaptation holds the potential to substantially improve the efficacy and engagement (i.e. promoting continued use) of eHealth applications. Pervasive technology suitable for eHealth applications (e.g. smartphones, in-home smart devices, etc.) are now common, and provide a rich dataset, covering much of daily life, and which may potentially be used for this adaptation. This approach has sometimes been referred to as “just-in-time adaptive interventions”, or JITAIs. One challenge in developing high-quality JITAIs is developing rules to control intervention and adaptation. For instance, rules are needed to identify an optimal time when a smartphone eHealth app should deliver a prompt to the user to take some action (e.g. physical activity).

Writing high-quality rules for JITAIs that perform well (i.e. reliably promote the desired behavior change or maintenance, and reliably maintain engagement and continued use of the intervention) for a wide variety of users is difficult, even for experts in health psychology or related fields. Individual users vary in many ways, both in terms of quasi-permanent traits (e.g. personality) and in change over time (e.g. varying emotional stress). Consequently, there are many possible variables to adapt on, including constructs identified by numerous theories of health behavior change (e.g. personality, self-efficacy, extrinsic and intrinsic motivation, etc.), and numerous potentially relevant measurements of the user and his/her environment (e.g. the user's fatigue, his/her daily schedule, his/her location and immediate context, etc.). Attempting to account for a growing number of these variables, and potential combinations of them, leads to a combinatorial explosion of possible rules. Consequently, hand-authored rules cannot feasibly account for more than a small fraction of the possible ways in which individual users differ from each other, and JITAIs based on hand-authored rules may have suboptimal effectiveness.

There is recent academic work on generic adaptive systems based on control theory and multi-arm bandit systems. However, these approaches also have challenges in implementation. For instance, such approaches are designed to provide alternatives (replacements) to an existing system, which makes them prone to an exploration phase when first interacting with a new user before gathering enough information for adapting interventions. During such an exploration phase, an adaptive system typically has very poor performance, making largely random choices of intervention actions which are unlikely to reliably promote behavior change, potentially leading to user resistance to future behavior change, or, worse, loss of user engagement and a refusal to continue using the intervention system.

### SUMMARY OF THE INVENTION

In one embodiment, a computer-implemented method, comprising: receiving contextual information for a user; updating a user state based on the received contextual information; providing electronic interventions to the user over a first interval by executing a first intervention algorithm based on the updated user state; and providing electronic interventions to the user over a second interval based on executing a second intervention algorithm that maximizes a reward function based on a further updated user state of the user and the electronic interventions of the first interval, the second intervention algorithm of a different type than the first intervention algorithm.

These and other aspects of the invention will be apparent from and elucidated with reference to the embodiment(s) described hereinafter.

### BRIEF DESCRIPTION OF THE DRAWINGS

Many aspects of the invention can be better understood with reference to the following drawings, which are diagrammatic. The components in the drawings are not necessarily to scale, emphasis instead being placed upon clearly illustrating the principles of the present invention. Moreover, in the drawings, like reference numerals designate corresponding parts throughout the several views.

FIG. 1 is a schematic diagram that illustrates an example environment in which an electronic health (eHealth) adaptive learning system is used in accordance with an embodiment of the invention.

FIG. 2 is a schematic diagram that illustrates an example wearable device in which all or a portion of the functionality of an eHealth adaptive learning system may be implemented, in accordance with an embodiment of the invention.

FIG. 3 is a schematic diagram that illustrates an example electronics device in which all or a portion of the functionality of an eHealth adaptive learning system may be implemented, in accordance with an embodiment of the invention.

FIG. 4 is a schematic diagram that illustrates an example computing system in which at least a portion of the functionality of an eHealth adaptive learning system may be implemented, in accordance with an embodiment of the invention.

FIG. 5 is a logical flow diagram that illustrates underlying logic of an eHealth adaptive learning system, in accordance with an embodiment of the invention.

FIG. 6 is a plot diagram that illustrates action selection probability over time for example operations of an eHealth adaptive learning system, in accordance with an embodiment of the invention.

FIG. 7 is a logical flow diagram that illustrates adaptation functionality for an eHealth adaptive learning system, in accordance with an embodiment of the invention.

FIG. 8 is a flow diagram that illustrates an example eHealth learning method, in accordance with an embodiment of the invention.

### DETAILED DESCRIPTION OF EMBODIMENTS

Disclosed herein are certain embodiments of an electronic health (eHealth) adaptive learning system, method, and computer readable medium (herein, also collectively referred to as an eHealth adaptive learning system) that augment existing behavior change programs by starting with prior interventions and adapting them based on a reinforcement learning algorithm, namely a Q function, that estimates the long term value of taking an action in the context of a particular user state. More specifically, certain embodiments of an eHealth adaptive learning system augments an eHealth intervention with a learning and adaptation module that applies reinforcement learning algorithms to automatically adapt to individual users to optimize a reward function specified by system operators, while obeying specified constraints to enforce guarantees on system behavior imposed for safety or regulatory reasons.

Digressing briefly, personalized behavioral coaching programs (e.g., eHealth applications, including those for healthy lifestyle promotion, chronic condition self-management, medication adherence, etc.) serve to improve the efficacy and engagement by a user. As indicated above, one way to personalize the coaching is by just-in-time adaptive interventions to help a user to take some action. However, existing approaches to adaptation rely on expert-written rules, which are limited by the difficulty of predicting and catering for all the possible situations in such rules and by the feasibility of extensive user assessment. Certain embodiments of an eHealth adaptive learning system address these and/or other intervention challenges based on several approaches. In one embodiment, an eHealth adaptive learning system augments, rather than replaces, an existing behavioral health intervention solution (e.g., typically one relying on expert-authored rules and lacking automated learning and adaptive features). This prior intervention is used initially while the system is learning about an individual user and enables the eHealth adaptive learning system to avoid problems of other approaches, including excessively slow adaptation and poor behavior while learning. Further, certain embodiments of an eHealth adaptive learning system uses reinforcement learning algorithms. Reinforcement learning algorithms comprise a subfield of machine learning that examines algorithms for choosing a sequence of actions, each of which has an unknown effect, to maximize some reward. Reinforcement learning algorithms are applicable to the learning and adaptation components of an eHealth adaptive learning system. Also, certain embodiments of an eHealth adaptive learning system use a particular class of reinforcement learning algorithms, namely, off-policy value function approximation, or what is more commonly referred to as Q-learning. Such learning algorithms are appropriate with modifications to performing learning and adaptation using an existing intervention (e.g., based on either hand-written rules or on previous use of the learning algorithm) as a starting point to improve early performance, and obey constraints to guarantee basic safety of an adaptive system for health-related applications.

Having summarized certain features of an eHealth adaptive learning system of the present disclosure, reference will now be made in detail to the description of an eHealth adaptive learning system as illustrated in the drawings. While an eHealth adaptive learning system will be described in connection with these drawings, there is no intent to limit notification systems to the embodiment or embodiments disclosed herein. For instance, an eHealth adaptive learning system may be used as a back-end component in a consumer-facing system, as a service to third parties, or as a decision support system for other programs, as explained further below. Further, although the description identifies or describes specifics of one or more embodiments, such specifics are not necessarily part of every embodiment, nor are all various stated advantages necessarily associated with a single embodiment or all embodiments. On the contrary, the intent is to cover all alternatives, modifications and equivalents included within the spirit and scope of the disclosure as defined by the appended claims. Further, it should be appreciated in the context of the present disclosure that the claims are not necessarily limited to the particular embodiments set out in the description.

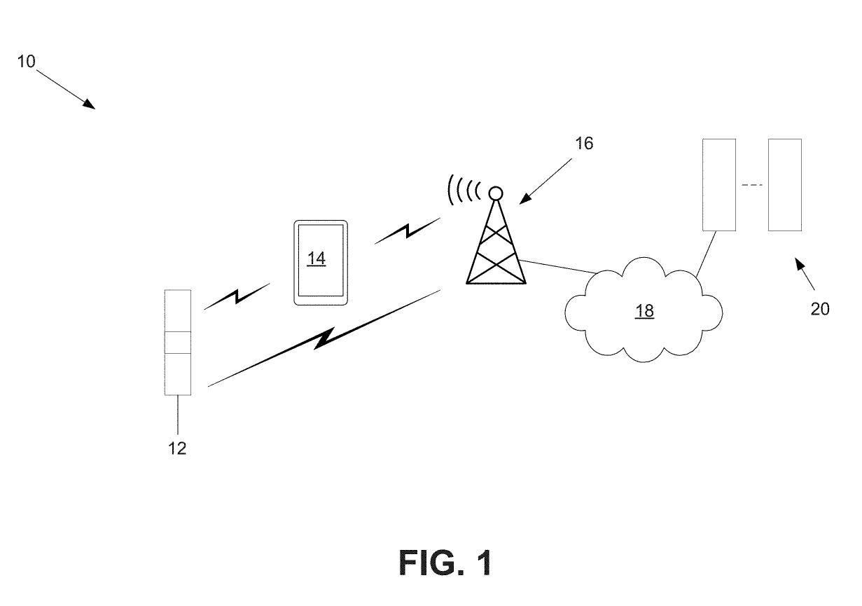

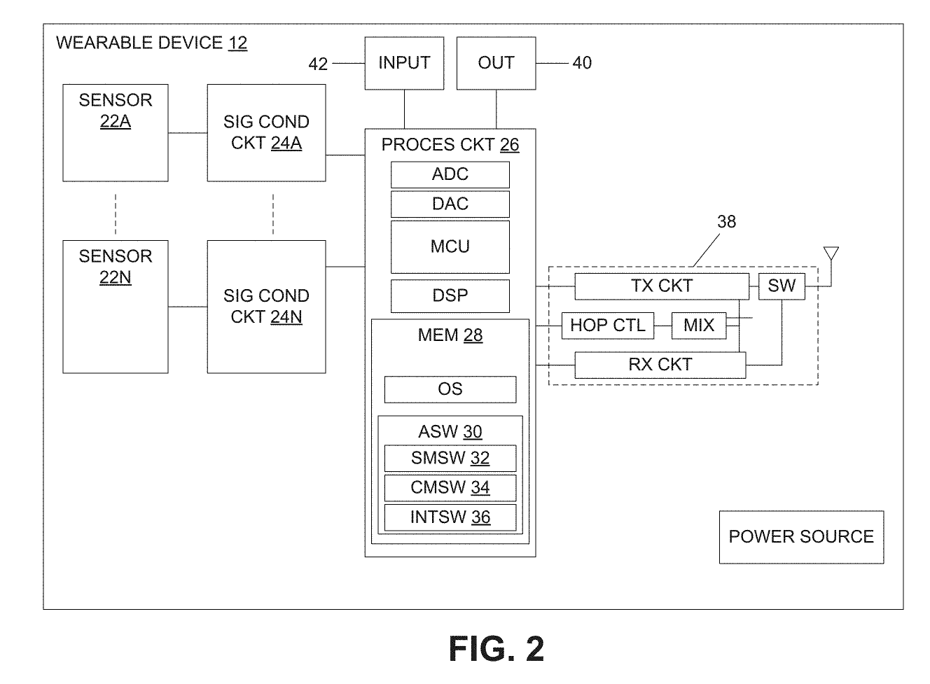

Referring now to FIG. 1, shown is an example environment 10 in which certain embodiments of an eHealth adaptive learning system may be implemented. It should be appreciated by one having ordinary skill in the art in the context of the present disclosure that the environment 10 is one example among many, and that some embodiments of an eHealth adaptive learning system may be used in environments with fewer, greater, and/or different components that those depicted in FIG. 1. The environment 10 comprises a plurality of devices that enable communication of information throughout one or more networks. The depicted environment 10 comprises a wearable device 12, an electronics (portable) device 14, a cellular network 16, a wide area network 18 (e.g., also described herein as the Internet), and a remote computing system 20. Note that the wearable device 12 and the electronics device 14 are also referred to as user devices. The wearable device 12, as described further in association with FIG. 2, is typically worn by the user (e.g., around the wrist or torso or attached to an article of clothing, or may include Google® glasses, wearable lenses, etc. using real time image capture, virtual, or augmented reality), and comprises a plurality of sensors that track physical activity of the user (e.g., steps, swim strokes, pedaling strokes, etc.), sense/measure or derive physiological parameters (e.g., heart rate, respiration, skin temperature, etc.) based on the sensor data, and optionally sense various other parameters (e.g., outdoor temperature, humidity, location, etc.) pertaining to the surrounding environment of the wearable device 12. For instance, in some embodiments, the wearable device 12 may comprise a global navigation satellite system (GNSS) receiver, including a GPS receiver, which tracks and provides location coordinates for the device 12. In some embodiments, the wearable device 12 may comprise indoor location technology, including beacons, RFID or other coded light technologies, WiFi, etc. Some embodiments of the wearable device 12 may include a motion or inertial tracking sensor, including an accelerometer and/or a gyroscope, providing movement data of the user. A representation of such gathered data may be communicated to the user via an integrated display on the wearable device 12 and/or on another device or devices.

Also, such data gathered by the wearable device 12 may be communicated (e.g., continually, periodically, and/or aperiodically, including upon request) to one or more electronics devices, such as the electronics device 14 or via the cellular network 16 to a device or devices of the computing system 20. Such communication may be achieved wirelessly (e.g., using near field communications (NFC) functionality, Blue-tooth functionality, 802.11-based technology, etc.) and/or according to a wired medium (e.g., universal serial bus (USB), etc.). Further discussion of the wearable device 12 is described below in association with FIG. 2.

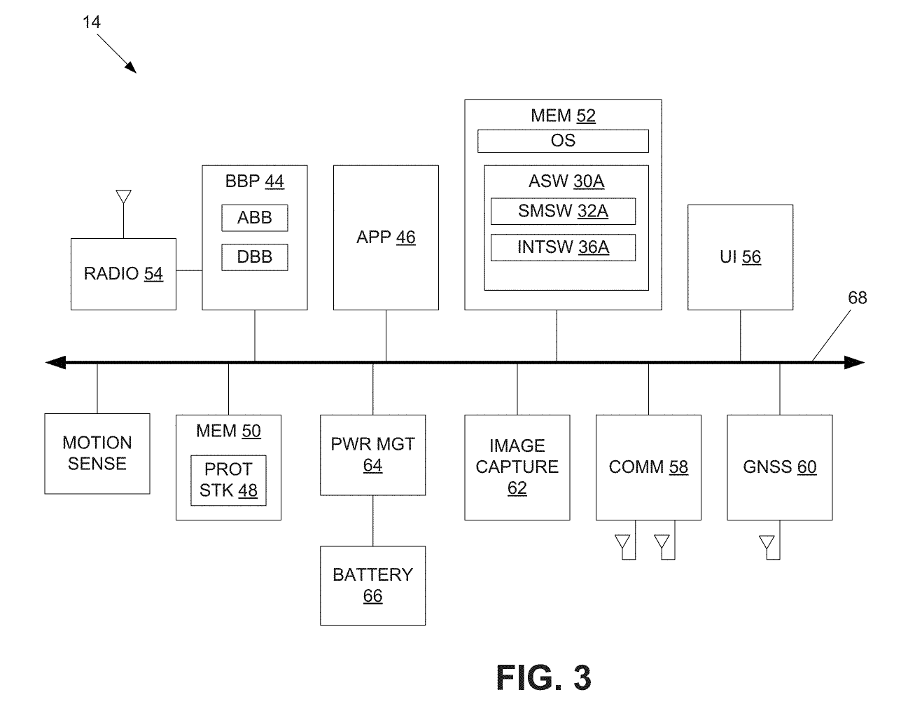

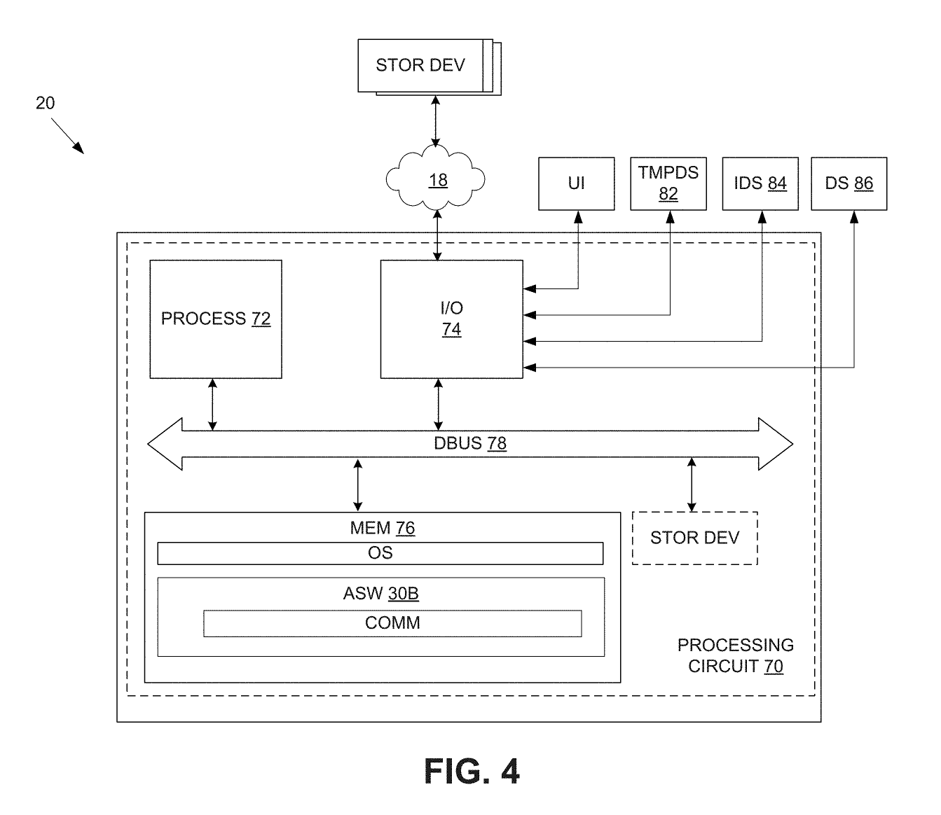

The electronics device 14 may be embodied as a smartphone, mobile phone, cellular phone, pager, stand-alone image capture device (e.g., camera), laptop, workstation, among other handheld and portable computing/communication devices, including communication devices having wireless communication capability, including telephony functionality. It is noted that if the electronics device 14 is embodied as a laptop or computer in general, the architecture more resembles that of the computing system 20 shown and described in association with FIG. 4. In some embodiments, the electronics device 14 may have built-in, image capture/recording functionality. In the depicted embodiment of FIG. 1, the electronics device 14 is a smartphone, though it should be appreciated that the electronics device 14 may take the form of other types of devices as described above. Further discussion of the electronics device 14 is described below in association with FIG. 3.

The cellular network 16 may include the necessary infrastructure to enable cellular communications by the electronics device 14 and optionally the wearable device 12. There are a number of different digital cellular technologies suitable for use in the cellular network 16, including: GSM, GPRS, CDMAOne, CDMA2000, Evolution-Data Optimized (EV-DO), EDGE, Universal Mobile Telecommunications System (UMTS), Digital Enhanced Cordless Telecommunications (DECT), Digital AMPS (IS-136/TDMA), and Integrated Digital Enhanced Network (iDEN), among others.

The wide area network 18 may comprise one or a plurality of networks that in whole or in part comprise the Internet. The electronics device 14 and optionally wearable device 12 access one or more of the devices of the computing system 20 via the Internet 18, which may be further enabled through access to one or more networks including PSTN (Public Switched Telephone Networks), POTS, Integrated Services Digital Network (ISDN), Ethernet, Fiber, DSL/ADSL, among others.

The computing system 20 comprises one or more devices coupled to the wide area network 18, including one or more computing devices networked together, including an application server(s) and data storage. The computing system 20 may serve as a cloud computing environment (or other server network) for the electronics device 14 and/or wearable device 12, performing processing and data storage on behalf of (or in some embodiments, in addition to) the electronics devices 14 and/or wearable device 12. In one embodiment, the computing system 20 may be configured to be a backend server for a health program. The computing system 20 receives observations (e.g., data) collected via sensors or input interfaces of one or more of the wearable device 12 or electronics device 14 and/or other devices or applications, stores the received data in a data structure (e.g., user profile database, etc.), and generates interventions (e.g., electronic interventions, including messages, notifications, or signals to activate haptic, light-emitting, or aural-based devices or hardware components, among other actions) for presentation to the user. The computing system 20 is programmed to handle the operations of one or more health or wellness programs implemented on the wearable device 12 and/or electronics device 14 via the networks 16 and/or 18. For example, the computing system 20 processes user registration requests, user device activation requests, user information updating requests, data uploading requests, data synchronization requests, etc. The data received at the computing system 20 may be a plurality of measurements pertaining to the parameters, for example, body movements and activities, heart rate, respiration rate, blood pressure, body temperature, light and visual information, etc., user feedback/input, and the corresponding context. Based on the data observed during a period of time and/or over a large population of users, the computing system 20 generates interventions pertaining to each specific parameter, and provides the interventions via the networks 16 and/or 18 for presentation on devices 12 and/or 14. In some embodiments, the computing system 20 is configured to be a backend server for a health-related program or a health-related application implemented on the mobile devices. The functions of the computing system 20 described above are for illustrative purpose only. The present disclosure is not intended to be limiting. The computing system 20 may be a general computing server or a dedicated computing server. The computing system 20 may be configured to provide backend support for a program developed by a specific manufacturer.

When embodied as a cloud service or services, the computing system 20 may comprise an internal cloud, an external cloud, a private cloud, or a public cloud (e.g., commercial cloud). For instance, a private cloud may be implemented using a variety of cloud systems including, for example, Eucalyptus Systems, VMWare vSphere®, or Microsoft® HyperV. A public cloud may include, for example, Amazon EC2®, Amazon Web Services®, Terremark®, Savvis®, or GoGrid®. Cloud-computing resources provided by these clouds may include, for example, storage resources (e.g., Storage Area Network (SAN), Network File System (NFS), and Amazon 53®), network resources (e.g., firewall, load-balancer, and proxy server), internal private resources, external private resources, secure public resources, infrastructure-as-a-services (IaaSs), platform-as-a-services (PaaSs), or software-as-a-services (SaaSs). The cloud architecture of the computing system 20 may be embodied according to one of a plurality of different configurations. For instance, if configured according to MICROSOFT AZURE™, roles are provided, which are discrete scalable components built with managed code. Worker roles are for generalized development, and may perform background processing for a web role. Web roles provide a web server and listen and respond for web requests via an HTTP (hypertext transfer protocol) or HTTPS (HTTP secure) endpoint. VM roles are instantiated according to tenant defined configurations (e.g., resources, guest operating system). Operating system and VM updates are managed by the cloud. A web role and a worker role run in a VM role, which is a virtual machine under the control of the tenant. Storage and SQL services are available to be used by the roles. As with other clouds, the hardware and software environment or platform, including scaling, load balancing, etc., are handled by the cloud.

In some embodiments, the computing system 20 may be configured into multiple, logically-grouped servers, referred to as a server farm. The computing system 20 may comprise plural server devices geographically dispersed, administered as a single entity, or distributed among a plurality of server farms, executing one or more applications on behalf of one or more of the devices 12 and/or 14. The devices of the computing system 20 within each farm may be heterogeneous. One or more of the devices of the computing system 20 may operate according to one type of operating system platform (e.g., WINDOWS NT, manufactured by Microsoft Corp. of Redmond, Wash.), while one or more of the other devices of the computing system 20 may operate according to another type of operating system platform (e.g., Unix or Linux). The devices of the computing system 20 may be logically grouped as a server farm that may be interconnected using a wide-area network (WAN) connection or medium-area network (MAN) connection. The devices of the computing system 20 may each be referred to as, and operate according to, a file server device, application server device, web server device, proxy server device, or gateway server device. In one embodiment, the computing system 20 provides an API or web interface that enables the devices 12 and/or 14 to communicate with the computing system 20. The computing system 20 may also be configured to be interoperable across other servers and generate statements in a format that is compatible with other programs. In some embodiments, one or more of the functionality of the computing system 20 may be performed at the respective devices 12 and/or 14. Further discussion of the computing system 20 is described below in association with FIG. 4.

An embodiment of an eHealth adaptive learning system may comprise the wearable device 12, the electronics device 14, and/or the computing system 20. In other words, one or more of the aforementioned devices 12, 14, and 20 may implement the functionality of an eHealth adaptive learning system. For instance, the wearable device 12 may comprise all of the functionality of an eHealth adaptive learning system, enabling the user to avoid the need for Internet connectivity and/or carrying a smartphone 14 around. In some embodiments, the functionality of the eHealth adaptive learning system may be implemented using any combination of the wearable device 12 and the electronics device 14 and/or the computing system 20. For instance, the wearable device 12 and/or the electronics device 14 may present interventions (e.g., electronic interventions/actions) via a user interface and provide sensing and/or input functionality with corresponding observations communicated to the computing system 20, which provides for processing of the observations and communication of the interventions based on the processing.

As an example, the wearable device 12 may monitor activity of the user, and communicate context and the sensed parameters (e.g., location coordinates, motion data, physiological data, etc.) to one of the devices (e.g., the electronics device 14 and/or the computing system 20) external to the wearable device 12. The electronics device 14 and/or wearable device 12 may be used to obtain other observations (e.g., inputs by users). Such observations that are sensed and/or otherwise acquired are communicated to the computing system 20 for processing and eventually, issuance of interventions. One benefit to the latter embodiment is that off-loading of the computational resources of the wearable device 12 and/or the electronics device 14 is enabled, conserving power consumed by the wearable device 12 and/or the electronics device 14. In some embodiments, the interventions may be presented by the wearable device 12 and/or the electronics device 14 and all other processing may be performed by the computing system 20, and in some embodiments, the interventions may be presented by the wearable device 12 and/or the electronics device 14 and all other processing performed by the electronics device 14, and in some embodiments, the interventions and processing may be entirely performed by the wearable device 12 and/or the electronics device 14. These and/or other variations are contemplated to be within the scope of the disclosure.

Attention is now directed to FIG. 2, which illustrates an example wearable device 12 in which all or a portion of the functionality of an eHealth adaptive learning system may be implemented. That is, FIG. 2 illustrates an example architecture (e.g., hardware and software) for the example wearable device 12. It should be appreciated by one having ordinary skill in the art in the context of the present disclosure that the architecture of the wearable device 12 depicted in FIG. 2 is but one example, and that in some embodiments, additional, fewer, and/or different components may be used to achieve similar and/or additional functionality. In one embodiment, the wearable device 12 comprises a plurality of sensors 22 (e.g., 22A-22N), one or more signal conditioning circuits 24 (e.g., SIG COND CKT 24A-SIG COND CKT 24N) coupled respectively to the sensors 22, and a processing circuit 26 (PROCES CKT) that receives the conditioned signals from the signal conditioning circuits 24. In one embodiment, the processing circuit 26 comprises an analog-to-digital converter (ADC), a digital-to-analog converter (DAC), a microcontroller unit (MCU), a digital signal processor (DSP), and memory (MEM) 28. In some embodiments, the processing circuit 26 may comprise fewer or additional components than those depicted in FIG. 2. For instance, in one embodiment, the processing circuit 26 may consist of the microcontroller. In some embodiments, the processing circuit 26 may include the signal conditioning circuits 24. The memory 28 comprises an operating system (OS) and application software (ASW) 30.

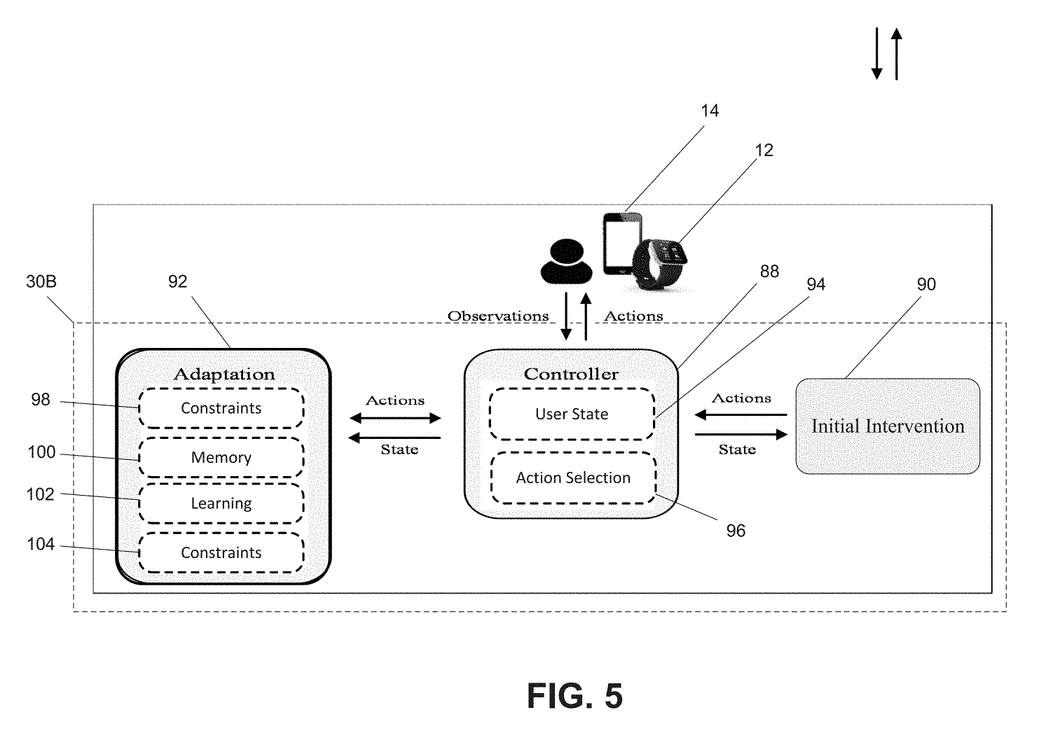

The application software 30 comprises a plurality of software modules (e.g., executable code/instructions), including an eHealth app and sensor measurement software (SMSW) 32, communications software (CMSW) 34, and interventions software (INTSW) 36, one or more of which may be part of the eHealth app. In some embodiments, the application software 30 may include additional software that implements some or all of the processing functionality of an eHealth adaptive learning system (as described further in association with FIG. 5). For purposes of brevity, the description about the application software 30 hereinafter is premised on the assumption that the various processing performed by the eHealth adaptive learning system is implemented at the computing device 20, and that the communication of observations (e.g., sensor data, user input, etc.) to the electronics device 14 and/or computing system 20 and the presentation of the interventions by the wearable device 12 is performed at the wearable device 12 (and/or electronics device 14, FIG. 1). The sensor measurement software 32 comprises executable code to process the signals (and associated data) measured by the sensors 22 and record and/or derive physiological parameters, such as heart rate, blood pressure, respiration, perspiration, etc. and movement and/or location data.

The communications software 34 comprises executable code/instructions to enable a communications circuit 38 of the wearable device 12 to operate according to one or more of a plurality of different communication technologies (e.g., NFC, Bluetooth, Wi-Fi, including 802.11, GSM, LTE, CDMA, WCDMA, Zigbee, etc.). The communications software 34 instructs and/or controls the communications circuit 38 to transmit the raw sensor data and/or the derived information (and/or other user data) from the sensor data to the computing system 20 (e.g., directly via the cellular network 16, or indirectly via the electronics device 14). The communications software 34 may also include browser software in some embodiments to enable Internet connectivity. The communications software 34 may also be used to access certain services, such as mapping/place location services, which may be used to determine context for the sensor data. These services may be used in some embodiments of an eHealth adaptive learning system, and in some instances, may not be used. In some embodiments, the communications software 34 may be external to the application software 30 or in other segments of memory. The interventions software 36 is configured to receive the interventions via the communications software 34 and communications circuit 38 as the interventions are communicated at different (e.g., non-overlapping) intervals based on the context (e.g., determined by the computing system 20 from the input data received from the wearable device 12 and/or the electronics device, among other devices). The interventions software 36 may format and present the interventions at an output interface 40 of the wearable device 12 at a time corresponding to when the interventions are received from the computing system 20 and/or electronics device 14 and/or at other times during the day or evening if different than when received. In some embodiments, the interventions software 36 may learn (e.g., based on previous interventions that were indicated, such as via feedback or use or neglect of similar and/or previous interventions) a preferred or best moment to present a current interventions received from the computing system 20. In some embodiments, this scheduling function may be performed by processing functionality at the computing system 20.

As indicated above, in one embodiment, the processing circuit 26 is coupled to the communications circuit 38. The communications circuit 38 serves to enable wireless communications between the wearable device 12 and other devices, including the electronics device 14 and the computing system 20, among other devices. The communications circuit 38 is depicted as a Bluetooth circuit, though not limited to this transceiver configuration. For instance, in some embodiments, the communications circuit 38 may be embodied as one or any combination of an NFC circuit, Wi-Fi circuit, transceiver circuitry based on Zigbee, 802.11, GSM, LTE, CDMA, WCDMA, among others such as optical or ultrasonic based technologies. The processing circuit 26 is further coupled to input/output (I/O) devices or peripherals, including an input interface 42 (INPUT) and the output interface 40 (OUT). Note that in some embodiments, functionality for one or more of the aforementioned circuits and/or software may be combined into fewer components/modules, or in some embodiments, further distributed among additional components/modules or devices. For instance, the processing circuit 26 may be packaged as an integrated circuit that includes the microcontroller (microcontroller unit or MCU), the DSP, and memory 28, whereas the ADC and DAC may be packaged as a separate integrated circuit coupled to the processing circuit 26. In some embodiments, one or more of the functionality for the above-listed components may be combined, such as functionality of the DSP performed by the microcontroller.

The sensors 22 are selected (e.g., by logic of the wearable device 12) to perform detection and measurement of a plurality of physiological and behavioral parameters (e.g., typical behavioral parameters or activities including walking, running, cycling, and/or other activities, including shopping, walking a dog, working in the garden, etc.), including heart rate, heart rate variability, heart rate recovery, blood flow rate, activity level, muscle activity (e.g., movement of limbs, repetitive movement, core movement, body orientation/position, power, speed, acceleration, etc.), muscle tension, blood volume, blood pressure, blood oxygen saturation, respiratory rate, perspiration, skin temperature, body weight, and body composition (e.g., body mass index or BMI). At least one of the sensors 22 may be embodied as movement detecting sensors, including inertial sensors (e.g., gyroscopes, single or multi-axis accelerometers, such as those using piezoelectric, piezoresistive or capacitive technology in a microelectromechanical system (MEMS) infrastructure for sensing movement) and/or as GNSS sensors, including a GPS receiver to facilitate determinations of distance, speed, acceleration, location, altitude, etc. (e.g., location data, or generally, sensing movement), in addition to or in lieu of the accelerometer/gyroscope and/or indoor tracking (e.g., ibeacons, WiFi, coded-light based technology, etc.). The sensors 22 may also include flex and/or force sensors (e.g., using variable resistance), electromyographic sensors, electrocardiographic sensors (e.g., EKG, ECG) magnetic sensors, photoplethysmographic (PPG) sensors, bio-impedance sensors, infrared proximity sensors, acoustic/ultrasonic/audio sensors, a strain gauge, galvanic skin/sweat sensors, pH sensors, temperature sensors, pressure sensors, and photocells. The sensors 22 may include other and/or additional types of sensors for the detection of, for instance, barometric pressure, humidity, outdoor temperature, etc. In some embodiments, GNSS functionality may be achieved via the communications circuit 38 or other circuits coupled to the processing circuit 26.

The signal conditioning circuits 24 include amplifiers and filters, among other signal conditioning components, to condition the sensed signals including data corresponding to the sensed physiological parameters and/or location signals before further processing is implemented at the processing circuit 26. Though depicted in FIG. 2 as respectively associated with each sensor 22, in some embodiments, fewer signal conditioning circuits 24 may be used (e.g., shared for more than one sensor 22). In some embodiments, the signal conditioning circuits 24 (or functionality thereof) may be incorporated elsewhere, such as in the circuitry of the respective sensors 22 or in the processing circuit 26 (or in components residing therein). Further, although described above as involving unidirectional signal flow (e.g., from the sensor 22 to the signal conditioning circuit 24), in some embodiments, signal flow may be bi-directional. For instance, in the case of optical measurements, the microcontroller may cause an optical signal to be emitted from a light source (e.g., light emitting diode(s) or LED(s)) in or coupled to the circuitry of the sensor 22, with the sensor 22 (e.g., photocell) receiving the reflected/refracted signals.

The communications circuit 38 is managed and controlled by the processing circuit 26 (e.g., executing the communications software 34). The communications circuit 38 is used to wirelessly interface with the electronics device 14 (FIG. 3) and/or one or more devices of the computing system 20. In one embodiment, the communications circuit 38 may be configured as a Bluetooth (including BTLE) transceiver, though in some embodiments, other and/or additional technologies may be used, such as Wi-Fi, GSM, LTE, CDMA and its derivatives, Zigbee, NFC, among others. In the embodiment depicted in FIG. 2, the communications circuit 38 comprises a transmitter circuit (TX CKT), a switch (SW), an antenna, a receiver circuit (RX CKT), a mixing circuit (MIX), and a frequency hopping controller (HOP CTL). The transmitter circuit and the receiver circuit comprise components suitable for providing respective transmission and reception of an RF signal, including a modulator/demodulator, filters, and amplifiers. In some embodiments, demodulation/modulation and/or filtering may be performed in part or in whole by the DSP. The switch switches between receiving and transmitting modes. The mixing circuit may be embodied as a frequency synthesizer and frequency mixers, as controlled by the processing circuit 26. The frequency hopping controller controls the hopping frequency of a transmitted signal based on feedback from a modulator of the transmitter circuit. In some embodiments, functionality for the frequency hopping controller may be implemented by the microcontroller or DSP. Control for the communications circuit 38 may be implemented by the microcontroller, the DSP, or a combination of both. In some embodiments, the communications circuit 38 may have its own dedicated controller that is supervised and/or managed by the microcontroller.

In one example operation, a signal (e.g., at 2.4 GHz) may be received at the antenna and directed by the switch to the receiver circuit. The receiver circuit, in cooperation with the mixing circuit, converts the received signal into an intermediate frequency (IF) signal under frequency hopping control attributed by the frequency hopping controller and then to baseband for further processing by the ADC. On the transmitting side, the baseband signal (e.g., from the DAC of the processing circuit 26) is converted to an IF signal and then RF by the transmitter circuit operating in cooperation with the mixing circuit, with the RF signal passed through the switch and emitted from the antenna under frequency hopping control provided by the frequency hopping controller. The modulator and demodulator of the transmitter and receiver circuits may be frequency shift keying (FSK) type modulation/demodulation, though not limited to this type of modulation/demodulation, which enables the conversion between IF and baseband. In some embodiments, demodulation/modulation and/or filtering may be performed in part or in whole by the DSP. The memory 28 stores the communications software 34, which when executed by the microcontroller, controls the Bluetooth (and/or other protocols) transmission/reception.

Though the communications circuit 38 is depicted as an IF-type transceiver, in some embodiments, a direct conversion architecture may be implemented. As noted above, the communications circuit 38 may be embodied according to other and/or additional transceiver technologies.

The processing circuit 26 is depicted in FIG. 2 as including the ADC and DAC. For sensing functionality, the ADC converts the conditioned signal from the signal conditioning circuit 24 and digitizes the signal for further processing by the microcontroller and/or DSP. The ADC may also be used to convert analogs inputs that are received via the input interface 42 to a digital format for further processing by the microcontroller. The ADC may also be used in baseband processing of signals received via the communications circuit 38. The DAC converts digital information to analog information. Its role for sensing functionality may be to control the emission of signals, such as optical signals or acoustic signal, from the sensors 22. The DAC may further be used to cause the output of analog signals from the output interface 40. Also, the DAC may be used to convert the digital information and/or instructions from the microcontroller and/or DSP to analog signals that are fed to the transmitter circuit. In some embodiments, additional conversion circuits may be used.

The microcontroller and the DSP provide the processing functionality for the wearable device 12. In some embodiments, functionality of both processors may be combined into a single processor, or further distributed among additional processors. The DSP provides for specialized digital signal processing, and enables an offloading of processing load from the microcontroller. The DSP may be embodied in specialized integrated circuit(s) or as field programmable gate arrays (FPGAs). In one embodiment, the DSP comprises a pipelined architecture, which comprises a central processing unit (CPU), plural circular buffers and separate program and data memories according to a Harvard architecture. The DSP further comprises dual busses, enabling concurrent instruction and data fetches. The DSP may also comprise an instruction cache and I/O controller, such as those found in Analog Devices SHARC® DSPs, though other manufacturers of DSPs may be used (e.g., Freescale multi-core MSC81xx family, Texas Instruments C6000 series, etc.). The DSP is generally utilized for math manipulations using registers and math components that may include a multiplier, arithmetic logic unit (ALU, which performs addition, subtraction, absolute value, logical operations, conversion between fixed and floating point units, etc.), and a barrel shifter. The ability of the DSP to implement fast multiply-accumulates (MACs) enables efficient execution of Fast Fourier Transforms (FFTs) and Finite Impulse Response (FIR) filtering. Some or all of the DSP functions may be performed by the microcontroller. The DSP generally serves an encoding and decoding function in the wearable device 12. For instance, encoding functionality may involve encoding commands or data corresponding to transfer of information to the electronics device 14 or a device of the computing system 20. Also, decoding functionality may involve decoding the information received from the sensors 22 (e.g., after processing by the ADC).

The microcontroller comprises a hardware device for executing software/firmware, particularly that stored in memory 28. The microcontroller can be any custom made or commercially available processor, a central processing unit (CPU), a semiconductor based microprocessor (in the form of a microchip or chip set), a macroprocessor, or generally any device for executing software instructions. Examples of suitable commercially available microprocessors include Intel's® Itanium® and Atom® microprocessors, to name a few non-limiting examples. The microcontroller provides for management and control of the wearable device 12, including determining physiological parameters and/or location coordinates based on the sensors 22, and for enabling communication with the electronics device 14 and/or a device of the computing system 20, and for the presentation of interventions.

The memory 28 can include any one or combination of volatile memory elements (e.g., random access memory (RAM, such as DRAM, SRAM, SDRAM, etc.)) and nonvolatile memory elements (e.g., ROM, Flash, solid state, EPROM, EEPROM, etc.). Moreover, the memory 28 may incorporate electronic, magnetic, and/or other types of storage media.

The software in memory 28 may include one or more separate programs, each of which comprises an ordered listing of executable instructions for implementing logical functions. In the example of FIG. 2, the software in the memory 28 includes a suitable operating system and the application software 30, which includes a plurality of software modules 32-36 for implementing certain embodiments of an eHealth adaptive learning system and algorithms for determining physiological and/or behavioral measures and/or other information (e.g., including location, speed of travel, etc.) based on the output from the sensors 22. The raw data from the sensors 22 may be used by the algorithms to determine various physiological and/or behavioral measures (e.g., heart rate, biomechanics, such as swinging of the arms), and may also be used to derive other parameters, such as energy expenditure, heart rate recovery, aerobic capacity (e.g., VO2 max, etc.), among other derived measures of physical performance. In some embodiments, these derived parameters may be computed externally (e.g., at the electronics devices 14 or one or more devices of the computing system 20) in lieu of, or in addition to, the computations performed local to the wearable device 12. In some embodiments, the GPS functionality of the sensors 22 collects contextual data (time and location data, including location coordinates). The application software 30 may collect location data by sampling the location readings from the sensor 22 over a period of time (e.g., minutes, hours, days, weeks, etc.). The application software 30 may also collect information about the means of ambulation. For instance, the GPS data (which may include time coordinates) may be used by the application software 30 to determine speed of travel, which may indicate whether the user is moving within a vehicle, on a bicycle, or walking or running. In some embodiments, other and/or additional data may be used to assess the type of activity, including physiological data (e.g., heart rate, respiration rate, galvanic skin response, etc.) and/or behavioral data.

The operating system essentially controls the execution of other computer programs, such as the application software 30 and associated modules 32-36, and provides scheduling, input-output control, file and data management, memory management, and communication control and related services. The memory 28 may also include user data, including weight, height, age, gender, goals, body mass index (BMI) that are used by the microcontroller executing the executable code of the algorithms to accurately interpret the measured physiological and/or behavioral data. The user data may also include historical data relating past recorded data to prior contexts.

Although the application software 30 (and component parts 32-36) are described above as implemented in the wearable device 12, some embodiments may distribute the corresponding functionality among the wearable device 12 and other devices (e.g., electronics device 14 and/or one or more devices of the computing system 20), or in some embodiments, the application software 30 (and component parts 32-36) may be implemented in another device (e.g., the electronics device 14).

The software in memory 28 comprises a source program, executable program (object code), script, or any other entity comprising a set of instructions to be performed. When a source program, then the program may be translated via a compiler, assembler, interpreter, or the like, so as to operate properly in connection with the operating system. Furthermore, the software can be written as (a) an object oriented programming language, which has classes of data and methods, or (b) a procedure programming language, which has routines, subroutines, and/or functions, for example but not limited to, C, C++, Python, Java, among others. The software may be embodied in a computer program product, which may be a non-transitory computer readable medium or other medium.

The input interface 42 comprises an interface (e.g., including a user interface) for entry of user input, such as a button or microphone or sensor (e.g., to detect user input) or touch-type display. In some embodiments, the input interface 42 may serve as a communications port for downloaded information to the wearable device 12 (such as via a wired connection). The output interfaces 40 comprises an interface for the presentation or transfer of data, including a user interface (e.g., display screen presenting a graphical user interface) or communications interface for the transfer (e.g., wired) of information stored in the memory, or to enable one or more feedback devices, such as lighting devices (e.g., LEDs), audio devices (e.g., tone generator and speaker), and/or tactile feedback devices (e.g., vibratory motor). For instance, the output interface 40 may be used to present the interventions to the user. In some embodiments, at least some of the functionality of the input and output interfaces 42 and 40, respectively, may be combined, including being embodied at least in part as a touch-type display screen for the entry of input (e.g., to provide feedback that is communicated to the electronics device 14 and/or the computing system 20, to select one or more options to effect behavioral change, such as via a presented dashboard or other screen, to input preferences, etc.) and presentation of interventions, among other data. In some embodiments, selection may be made automatically after the invitation or prompt based on detecting the context of the user (e.g., a context aware feature).

Referring now to FIG. 3, shown is an example electronics device 14 in which all or a portion of the functionality of an eHealth adaptive learning system may be implemented. Similar to the description for the wearable device 12 of FIG. 2, and for the sake of brevity, the application software of the electronics device 14 comprises similar components as that for the wearable device 12, with the understanding that fewer or a greater number of software modules of the eHealth adaptive learning system may be used in some embodiments. In the depicted example, the electronics device 14 is embodied as a smartphone (hereinafter, referred to smartphone 14), though in some embodiments, other types of devices may be used, such as a workstation, laptop, notebook, tablet, etc. It should be appreciated by one having ordinary skill in the art that the logical block diagram depicted in FIG. 3 and described below is one example, and that other designs may be used in some embodiments. The application software (ASW) 30A comprises a plurality of software modules (e.g., executable code/instructions) including an eHealth app and sensor measurement software (SMSW) 32A and intervention software (INTSW) 36A, as well as communications software as is expected of mobile phones. Note that the application software 30A (and component parts 32A and 36A) comprise at least some of the functionality of the application software 30 (and component parts 32 and 36) described above for the wearable device 12, and may include additional software pertinent to smartphone operations (e.g., possibly not found in wearable devices 12).

The smartphone 14 comprises at least two different processors, including a baseband processor (BBP) 44 and an application processor (APP) 46. As is known, the baseband processor 44 primarily handles baseband communication-related tasks and the application processor 46 generally handles inputs and outputs and all applications other than those directly related to baseband processing. The baseband processor 44 comprises a dedicated processor for deploying functionality associated with a protocol stack (PROT STK) 48, such as a GSM (Global System for Mobile communications) protocol stack, among other functions. The application processor 46 comprises a multi-core processor for running applications, including all or a portion of the application software 30A and its corresponding component parts 32A and 36A as described above in association with the wearable device 12 of FIG. 2. The baseband processor 44 and application processor 46 have respective associated memory (e.g., MEM) 50, 52, including random access memory (RAM), Flash memory, etc., and peripherals, and a running clock.

More particularly, the baseband processor 44 may deploy functionality of the protocol stack 48 to enable the smartphone 14 to access one or a plurality of wireless network technologies, including WCDMA (Wideband Code Division Multiple Access), CDMA (Code Division Multiple Access), EDGE (Enhanced Data Rates for GSM Evolution), GPRS (General Packet Radio Service), Zigbee (e.g., based on IEEE 802.15.4), Bluetooth, Wi-Fi (Wireless Fidelity, such as based on IEEE 802.11), and/or LTE (Long Term Evolution), among variations thereof and/or other telecommunication protocols, standards, and/or specifications. The baseband processor 44 manages radio communications and control functions, including signal modulation, radio frequency shifting, and encoding. The baseband processor 44 comprises, or may be coupled to, a radio (e.g., RF front end) 54 and/or a GSM modem having one or more antennas, and analog and digital baseband circuitry (ABB, DBB, respectively in FIG. 3). The radio 54 comprises a transceiver and a power amplifier to enable the receiving and transmitting of signals of a plurality of different frequencies, enabling access to the cellular network 16 (FIG. 1), and hence the communication of user data and associated contexts to the computing system 20 (FIG. 1) and the receipt of interventions from the computing system 20.

The analog baseband circuitry is coupled to the radio 54 and provides an interface between the analog and digital domains of the GSM modem. The analog baseband circuitry comprises circuitry including an analog-to-digital converter (ADC) and digital-to-analog converter (DAC), as well as control and power management/distribution components and an audio codec to process analog and/or digital signals received indirectly via the application processor 46 or directly from the smartphone user interface 56 (e.g., microphone, earpiece, ring tone, vibrator circuits, etc.). The ADC digitizes any analog signals for processing by the digital baseband circuitry. The digital baseband circuitry deploys the functionality of one or more levels of the GSM protocol stack (e.g., Layer 1, Layer 2, etc.), and comprises a microcontroller (e.g., microcontroller unit or MCU, also referred to herein as a processor) and a digital signal processor (DSP, also referred to herein as a processor) that communicate over a shared memory interface (the memory comprising data and control information and parameters that instruct the actions to be taken on the data processed by the application processor 46). The MCU may be embodied as a RISC (reduced instruction set computer) machine that runs a real-time operating system (RTIOS), with cores having a plurality of peripherals (e.g., circuitry packaged as integrated circuits) such as RTC (real-time clock), SPI (serial peripheral interface), I2C (inter-integrated circuit), UARTs (Universal Asynchronous Receiver/Transmitter), devices based on IrDA (Infrared Data Association), SD/MMC (Secure Digital/Multimedia Cards) card controller, keypad scan controller, and USB devices, GPRS crypto module, TDMA (Time Division Multiple Access), smart card reader interface (e.g., for the one or more SIM (Subscriber Identity Module) cards), timers, and among others. For receive-side functionality, the MCU instructs the DSP to receive, for instance, in-phase/quadrature (I/Q) samples from the analog baseband circuitry and perform detection, demodulation, and decoding with reporting back to the MCU. For transmit-side functionality, the MCU presents transmittable data and auxiliary information to the DSP, which encodes the data and provides it to the analog baseband circuitry (e.g., converted to analog signals by the DAC).

The application processor 46 operates under control of an operating system (OS) that enables the implementation of a plurality of user applications, including the application software 30A. The application processor 46 may be embodied as a System on a Chip (SOC), and supports a plurality of multimedia related features including web browsing to access one or more computing devices of the computing system 20 (FIG. 4) that are coupled to the Internet, email, multimedia entertainment, games, etc. For instance, the application processor 46 may execute interface software (e.g., middleware, such as a browser with or operable in association with one or more application program interfaces (APIs)) to enable access to a cloud computing framework or other networks to provide remote data access/storage/processing, and through cooperation with an embedded operating system, access to calendars, location services, reminders, etc. For instance, in some embodiments, the eHealth adaptive learning system may operate using cloud computing, where the processing of sensor data (e.g., location data, including data received from the wearable device 12 or from integrated sensors within the smartphone 14, including motion sense, image capture, location detect, or user input, etc.) and context may be achieved by one or more devices of the computing system 20. The application processor 46 generally comprises a processor core (Advanced RISC Machine or ARM), and further comprises or may be coupled to multimedia modules (for decoding/encoding pictures, video, and/or audio), a graphics processing unit (GPU), communication interfaces (COMM) 58, and device interfaces. The communication interfaces 58 may include wireless interfaces, including a Bluetooth (BT) (and/or Zigbee in some embodiments) module that enables wireless communication with an electronics device, including the wearable device 12, other electronics devices, and a Wi-Fi module for interfacing with a local 802.11 network. The application processor 46 further comprises, or is coupled to, a global navigation satellite systems (GNSS) transceiver or receiver (GNSS) 60 for access to a satellite network to, for instance, provide location services.

The device interfaces coupled to the application processor 46 may include the user interface 56, including a display screen. The display screen, similar to a display screen of the wearable device user interface, may be embodied in one of several available technologies, including LCD or Liquid Crystal Display (or variants thereof, such as Thin Film Transistor (TFT) LCD, In Plane Switching (IPS) LCD)), light-emitting diode (LED)-based technology, such as organic LED (OLED), Active-Matrix OLED (AMOLED), or retina or haptic-based technology. For instance, the display screen may be used to present web pages, dashboards, interventions, and/or other documents or data received from the computing system 20 and/or the display screen may be used to present information (e.g., interventions) in graphical user interfaces (GUIs) rendered locally in association with the application software 30A. The display screen may be used to render wearable sensor data. Other user interfaces 56 include a keypad, microphone, speaker, ear piece connector, I/O interfaces (e.g., USB (Universal Serial Bus)), SD/MMC card, among other peripherals. Also coupled to the application processor 46 is an image capture device (IMAGE CAPTURE) 62. The image capture device 62 comprises an optical sensor (e.g., a charged coupled device (CCD) or a complementary metal-oxide semiconductor (CMOS) optical sensor). The image capture device 62 may be used to detect various physiological parameters of a user, including blood pressure based on remote photoplethysmography (PPG). Also included is a power management device 64 that controls and manages operations of a battery 66. The components described above and/or depicted in FIG. 3 share data over one or more busses, and in the depicted example, via data bus 68. It should be appreciated by one having ordinary skill in the art, in the context of the present disclosure, that variations to the above may be deployed in some embodiments to achieve similar functionality.

In the depicted embodiment, the application processor 46 runs the application software 30A, which in one embodiment, includes a plurality of software modules (e.g., executable code/instructions) including an eHealth app and the sensor measurement software (SMSW) 32A and the intervention software (INTSW) 36A. Since the description of the application software 30 and software modules 32 and 36 has been described above in association with the wearable device 12 (FIG. 2), and since the same functionality is present in software 32A (albeit in some instances based on perhaps different sensor data) and 36A, discussion of the same here is omitted for brevity. It is noteworthy, however, that some or all of the software functionality may be implemented in the smartphone 14. For instance, all of the functionality of the application software 30A may be implemented in the smartphone 14, or functionality of the application software 30A may be divided among plural devices of the environment 10 (FIG. 1) in some embodiments. The application software 30A may also comprise executable code/instructions to process the signals (and associated data) measured by the sensors (of the wearable device 12 as communicated to the smartphone 14, or based on sensors integrated within the smartphone 14) and record and/or derive physiological parameters, such as heart rate, blood pressure, respiration, perspiration, etc. Note that functionality of the software modules 32A and 36A, similar to those described for the wearable device 12, may be combined in some embodiments, or further distributed among additional modules. In some embodiments, the execution of the application software 30A and associated modules 32A and 36A may be distributed among plural devices, as set forth above. Note that all or a portion of the aforementioned hardware and/or software of the smartphone 14 may be referred to herein as a processing circuit.

Referring now to FIG. 4, shown is a computing system 20 in which at least a portion of the functionality of an eHealth adaptive learning system may be implemented. The computing system 20 may comprise a single computing device as shown here, or in some embodiments, may comprise plural devices that at least collectively perform the functionality described below, among optionally other functions. In one embodiment, the computing system 20 may be embodied as an application server, a computer, among other computing devices. One having ordinary skill in the art should appreciate in the context of the present disclosure that the example computing system 20 is merely illustrative of one embodiment, and that some embodiments of computing devices may comprise fewer or additional components, and/or some of the functionality associated with the various components depicted in FIG. 4 may be combined, or further distributed among additional modules or computing devices, in some embodiments. The computing system 20 is depicted in this example as a computer system, including one providing a function of an application server. It should be appreciated that certain well-known components of computer systems are omitted here to avoid obfuscating relevant features of the computing system 20. In one embodiment, the computing system 20 comprises a processing circuit 70 (also referred to herein as a processing system) comprising hardware and software components. In some embodiments, the processing circuit 70 may comprise additional components or fewer components. For instance, memory may be separate. The processing circuit 70 comprises one or more processors, such as processor 72 (PROCESS), input/output (I/O) interface(s) 74 (I/O), and memory 76 (MEM), all coupled to one or more data busses, such as data bus 78 (DBUS). The memory 76 may include any one or a combination of volatile memory elements (e.g., random-access memory RAM, such as DRAM, and SRAM, etc.) and nonvolatile memory elements (e.g., ROM, Flash, solid state, EPROM, EEPROM, hard drive, tape, CDROM, etc.).

The memory 76 may store a native operating system (OS), one or more native applications, emulation systems, or emulated applications for any of a variety of operating systems and/or emulated hardware platforms, emulated operating systems, etc. In some embodiments, the processing circuit 70 may include, or be coupled to, one or more separate storage devices. For instance, in the depicted embodiment, the processing circuit 70 is coupled via the I/O interfaces 74 to template data structures (TMPDS) 82 and intervention data structures (IDS) 84, and further to data structures (DS) 86, each coupled to the I/O devices 74 (directly or via network storage) or the data bus 78 (e.g., via storage device).

In some embodiments, the template data structures 82, intervention data structures 84, and/or data structures 86 may be coupled to the processing circuit 70 via the data bus 78 or coupled to the processing circuit 70 via the I/O interfaces 74 as network-connected storage devices (STOR DEVS). The data structures 82, 84, and/or 86 may be stored in persistent memory (e.g., optical, magnetic, and/or semiconductor memory and associated drives). In some embodiments, the data structures 82, 84, and/or 86 may be stored in memory 76.

The template data structures 82 are configured to store one or more templates that are used in an intervention definition stage to generate the interventions conveying information to the user. The intervention for different objectives may use different templates. For example, education related interventions may apply templates with referral links to educational resources, feedback on performance may apply templates with rating/ranking comments, etc. The template data structures 82 may be maintained by an administrator operating the computing system 20. The template data structures 82 may be updated based on the usage of each template, the feedback on each generated intervention, etc. The templates that are more often used and/or receive more positive feedbacks from the users may be highly recommended to generate the interventions in the future. In some embodiments, the templates may be general templates that can be used to generate all types of interventions. In some other embodiments, the templates may be classified into categories, each category pertaining to a parameter. For example, templates for generating interventions pertaining to heart rate may be partially different from templates for generating interventions pertaining to sleep quality. The intervention data structures 84 are configured to store the interventions that are constructed based on the templates. The data structures 86 are configured to store user profile data including the real-time measurements of parameters for a large population of users, personal information of the large population of users, user-entered input, etc. In some embodiments, the data structures 86 are configured to store health-related information of the user. The data structures 86 may be a backend database of the computing system 20. In some embodiments, however, the data structures 86 may be in the form of network storage and/or cloud storage directly connected to the network 18 (FIG. 1). In some embodiments, the data structures 86 may serve as backend storage of the computing system 20 as well as network storage and/or cloud storage. The data structures 86 are updated periodically, aperiodically, and/or in response to a request from the wearable device 12, the electronics device 14, and/or the operations of the computing system 20.

In the embodiment depicted in FIG. 4, the memory 76 comprises an operating system (OS) and the application software 30B. In some embodiments, an operating system may be omitted. The application software 30B comprises one or more software modules that enables the functionality of the eHealth adaptive learning system, including one or more eHealth applications and the components/modules shown in FIG. 5 and described below. The application software 30B further comprises a communications module (COMM). The communications module formats the interventions to be issued according to one or any combination of human-perceivable format (e.g., visually, audibly, using tactile feedback, including Braille, etc.). In one embodiment, the communications module may comprise card presentation functionality. As used herein, content cards generated for a specific parameter define a family of interventions associated with the specific parameter. For example, the content cards generated for sleep quality define a family of interventions related to sleep quality, while the content cards generated for running define a family of interventions related to running. The content cards may be configured to present one intervention per card, though in some embodiments, additional interventions may be presented. Different families may define a different numbers of interventions for presentation. In some embodiments, the content cards may be configured to present respective interventions related to the feedback of an activity performance. In some embodiments, the content cards may be configured to present interventions comprising educational information, or medication information, etc. In some embodiments, the content cards may be configured to present respective interventions comprising insightful analysis of the user's health-related conditions. In some embodiments, the content cards may comprise only text statements. In some embodiments, the content cards may comprise content in multiple formats including but not limited to text, audio, video, flash, hyperlink to other sources, etc. It should be appreciated that the content cards may be generated for purposes other than the examples described above, and the format of the content cards may be adjustable for presentation on different user devices. The examples set forth above are for illustrative purposes; and the present disclosure is not intended to be limiting. For instance, presentation of the interventions is not limited to content card formats.

In one embodiment, the communications module is configured to receive the interventions, prepare the presentation of the content cards based on settings pre-defined by the user and/or the configuration of each individual user device. The settings pre-defined by the user may comprise how the user wants to be notified with the content cards, for example, in a text format, in a chart format, in an audio format with low-tone female voice, in a video/flash format, and/or the combinations thereof. The settings pre-defined by the user may further comprise when and how often the user wants to be notified with the content cards, for example, every evening around 9:00 pm, every afternoon after exercise, every week, every month, in real-time, and/or the combination thereof. The settings pre-defined by the user may further comprise a preferred user device to receive the content card if the user has multiple devices. The configuration of each individual user device may include the size and resolution of the display screen of a user device, the caching space of the user device, etc. In some embodiments, the communications module may determine the connection status of the user device before sending the content cards. If the user device is determined to be unavailable due to power off, offline, damaged, etc., the communications module may store the generated content card in memory 76 and/or upload the generated content card to the data structures 86. Once the user is detected logged-in using one of his or her user devices, the generated content card is transmitted to the user device for presentation. In some embodiments, if the preferred user device is unavailable, the communications module adjusts the content card for presentation in the logged-in user device.

The communications module further enables communications among network-connected devices and provides web and/or cloud services, among other software such as via one or more APIs. For instance, the communications module may receive (via I/O interfaces 74) input data (e.g., a content feed) from the wearable device 12 and/or the electronics device 14 that includes sensed data and a context for the sensed data, data from third-party databases (e.g., medical data base), data from social media, data from questionnaires, data from external devices (e.g., weight scales, environmental sensors, etc.), among other data. The content feed may be continual, intermittent, and/or scheduled. The communications module operates in conjunction with the I/O interfaces 74 to provide the interventions to the wearable device 12 and/or the electronics device 14.

Execution of the application software 30B may be implemented by the processor 72 under the management and/or control of the operating system. The processor 72 may be embodied as a custom-made or commercially available processor, a central processing unit (CPU) or an auxiliary processor among several processors, a semiconductor based microprocessor (in the form of a microchip), a macroprocessor, one or more application specific integrated circuits (ASICs), a plurality of suitably configured digital logic gates, and/or other well-known electrical configurations comprising discrete elements both individually and in various combinations to coordinate the overall operation of the computing system 20.

The I/O interfaces 74 comprise hardware and/or software to provide one or more interfaces to the Internet 18, as well as to other devices such as a user interface (UI) (e.g., keyboard, mouse, microphone, display screen, etc.) and/or the data structures 82,84, 86. The user interfaces may include a keyboard, mouse, microphone, immersive head set, display screen, etc., which enable input and/or output by an administrator or other user. The I/O interfaces 74 may comprise any number of interfaces for the input and output of signals (e.g., analog or digital data) for conveyance of information (e.g., data) over various networks and according to various protocols and/or standards. The user interface (UI) is configured to provide an interface between an administrator or content author and the computing system 20. The administrator may input a request via the user interface, for instance, to manage the template database 82. Upon receiving the request, the processor 72 instructs a template building component to process the request and provide information to enable the administrator to create, modify, and/or delete the templates.

When certain embodiments of the computing system 20 are implemented at least in part with software (including firmware), as depicted in FIG. 4, it should be noted that the application software 30B can be stored on a variety of non-transitory computer-readable medium for use by, or in connection with, a variety of computer-related systems or methods. In the context of this document, a computer-readable medium may comprise an electronic, magnetic, optical, or other physical device or apparatus that may contain or store a computer program (e.g., executable code or instructions) for use by or in connection with a computer-related system or method. The software may be embedded in a variety of computer-readable mediums for use by, or in connection with, an instruction execution system, apparatus, or device, such as a computer-based system, processor-containing system, or other system that can fetch the instructions from the instruction execution system, apparatus, or device and execute the instructions.

When certain embodiments of the computing system 20 are implemented at least in part with hardware, such functionality may be implemented with any or a combination of the following technologies, which are all well-known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), relays, contactors, etc.

Referring now to FIG. 5, and with continued reference to FIG. 4, shown is a logical flow diagram that illustrates the underlying logic and data flow of an eHealth adaptive learning system, or more particularly, the underlying logic of the application software 30B of the computing system 20. Though explained as a component of the computing system software, in some embodiments, the same or similar functionality may reside in the wearable device 12 and/or the electronics device 14, and in some embodiments, functionality of the application software 30B may be distributed among plural devices of the computing system 20 or plural devices located in disparate locations. The application software 30B is depicted in FIG. 5 as communicating with the wearable device 12 and electronic device 14, and in the description that follows for FIG. 5, communications (e.g., intervention actions and observations) to, and reception from, the devices 12 and/or 14 will collectively be referred to as communications to and/or from the user. The application software 30B comprises a controller module 88 (controller), a prior intervention module 90 (prior intervention), and an adaptation module 92 (adaptation). The controller module 88 comprises a user state module 94 (user state) and an action selection module 96 (action selection). The adaptation module 92 comprises a reward module 98 (reward), an experience memory module (memory) 100, a value learning module 102 (learning), and a constraints module 104 (constraints). The controller module 88 communicates with the user (e.g., via an existing behavioral intervention interface, such as a smartphone app), exchanging intervention actions (e.g. prompts to the user) and observations (e.g. measurements or self-report). The user state module 94 maintains a state summarizing relevant knowledge of the user and his/her environment, and updates this state when observations are received. The action selection module 96 chooses intervention actions to be sent to the user, selecting from among actions received from the prior intervention module 90 and from the adaptation module 92. Note that in some embodiments, the prior intervention module 90 may comprise one of the data structures, such as data structures 86 or 84, whether stored in one or more devices that are accessed via the I/O interfaces 74, or in memory 76. The prior intervention module 90 encapsulates an existing (typically rule-based) intervention, and executes it when requested by the controller module 88. Initially the prior intervention module 90 (and corresponding interventions) is used frequently, providing good initial performance and improving learning while the eHealth adaptive learning system (e.g., the application software 30B and processing circuit 70) is adapting to an individual user, and the use of the prior intervention is gradually decreased over time. The adaptation module 92 receives user state updates from the controller module 88, as well as intervention actions performed by the controller module 88, and responds with intervention actions chosen adaptively, maintaining and updating a model learned over time. The reward module 98 computes a reward or penalty received by the learning agent upon taking an intervention action, receiving subsequent observations from the user and/or environment, and inferring resulting changes to the user state module 94. The experience memory module 100 records user states observed, intervention actions performed, and rewards/penalties received, and makes this information available for use in learning. The value learning module 102 maintains an estimate of the “Q function”, or the long-term value (predicted cumulative average reward) from taking an action, depending on the current user state, and uses this estimate to select intervention actions. The constraints module 104 assists with enforcing restrictions on what intervention actions may be performed, enabling the eHealth adaptive learning system to guarantee safety and performance requirements even when its actions for an individual user are not fully predictable.

Explaining the above-described modules of the application software 30B further, the action selection module 96 receives observations from the user and/or environment, suggested actions from the prior intervention module 90 and the adaptation module 92, and outputs chosen actions to be delivered to the user. Note that actions, interventions, and intervention actions are used herein interchangeably. In one embodiment, a simple approach comprising a variant of the epsilon-greedy method, with linear annealing, is used. In particular, the action selection module 96 selects, each time an action is needed, randomly between the action produced by the adaptation module 92 and the action produced by the prior intervention module 90. Initially the probability of selecting the prior intervention action is high, but after an initial learning period, it decreases linearly, and the adapted action is selected more frequently. The action selection method of the action selection module 96 may be described using the following pseudocode:

```
Inputs:
  aₚ (prior intervention action)
  a_{L} (learned action)
  t (time since start of intervention)
Parameters:
  t₀ (start of adaptation period)
  p₀ (initial probability of prior action)
  p₁ (final probability of prior action)
  k (rate of adaptation)
Outputs:
  a (selected action)
Method:
    if t ≤ t₀:
      p = p₀
    else:
      p = max(p₁, p₀ − k*(t − t₀))
    if random (p):
    a = aₚ
  else:
    a = a_{L}
```

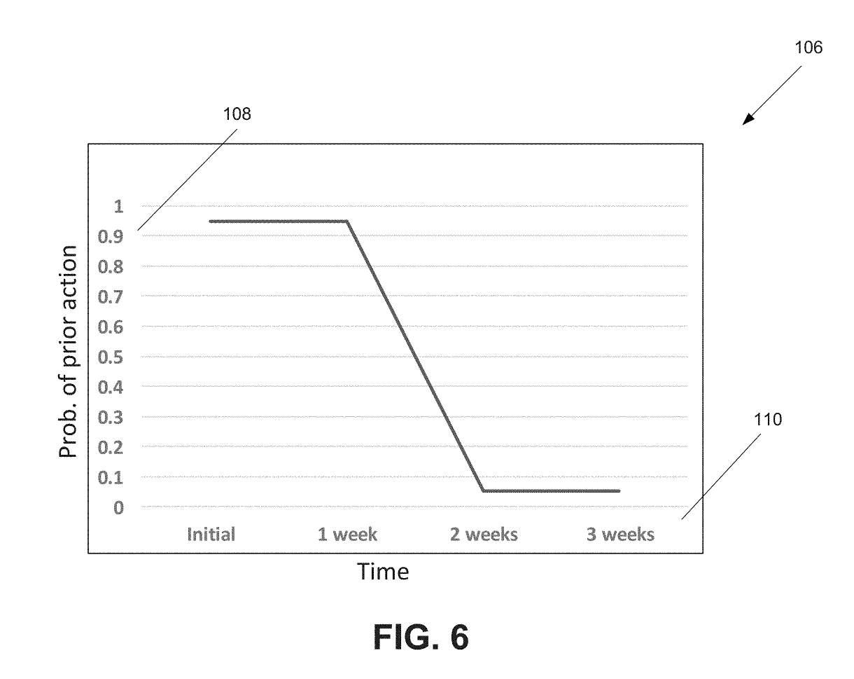

The tuning parameters (t₀, p₀, p₁, k) should be selected for individual use cases and to control the trade-off between the speed and robustness of adaptation. FIG. 6 is a plot diagram 106 that illustrates action selection probability over time for example operations of an eHealth adaptive learning system (e.g., shows the probability of action selection over time for a typical use case). The vertical axis 108 is the probability of prior action, and the horizontal axis 110 is time (e.g., initial, 1 week, 2 weeks, etc.). In this example, the probability of prior action selection is at 90% (0.9) from initial time to approximately 1 week, and starts to fall, reaching approximately zero probability in 2 weeks. Note that the results are merely illustrative of one example, and that performance may vary depending on the use case, among other factors.

Returning to FIG. 5, the user state module 94 maintains a user state, summarizing relevant features of the user and his/her environment. The current state is in turn transmitted to the prior intervention module 90 and the adaptation module 92. To apply a behavior change engine for a use case, the system operators should specify the set of features that makes up the user state and rules for updating those features when observations arrive from the user and/or the environment.

The prior intervention module 90 encapsulates an existing intervention, which is specified by the system operator. The eHealth adaptive learning system is flexible, and may use any component capable of selecting an intervention action given a user state. Possible prior interventions include random-selected actions, expert-written rules, a previously learned model (e.g., produced by the adaptation module 92) trained on experience with previous users and/or with simulated users.

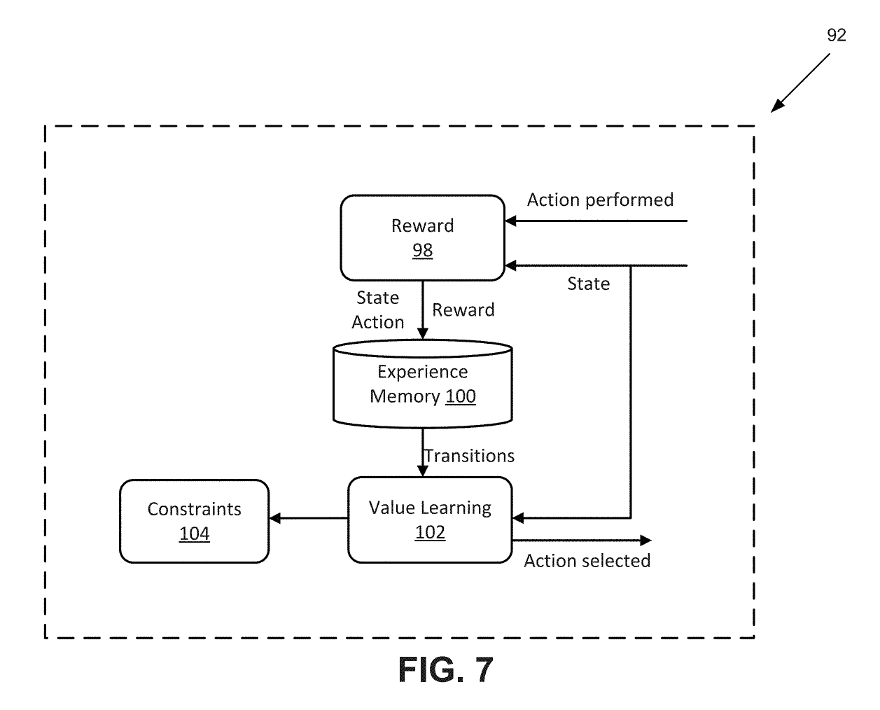

With continued reference to FIG. 5, attention is directed to FIG. 7, which comprises a logical flow diagram that illustrates adaptation functionality for an embodiment of an eHealth adaptive learning system. In particular, FIG. 7 shows an example internal organization and subcomponents of the adaptation module 92. Arrows to the right side of the diagram represent information flow to and from the controller module 88, as shown in FIG. 5. The reward module 98 determines the immediate reward or penalty received by the agent upon taking an intervention action (regardless of whether the action was chosen by the adaptation module 92 or by the prior intervention module 90). In one embodiment, the reward module 98 executes (e.g., the processing system 70 executing the reward module 98) a reward function specified by system operators, and tailored to a specific behavior change use case, which takes as inputs the user state prior to the last action, the last action, the current user state, the time of the last action, and/or the current time.

The experience memory module 100 records the state observed, action performed, and rewards received by the agent, and makes this information available for future learning. In one embodiment, the experience memory module 100 is implemented as a data structure (e.g., database) storing a time-indexed sequence of transitions, each of which includes the state immediately before an action, the action, the state after one unit of time has elapsed (e.g., with the unit of time specified by system operators and specific to a behavior change use case), and/or the reward received. The database is organized to support efficient querying of recent transitions within a window of time, or of a random subset of recent transitions.

The value learning module 102 maintains an estimate of the Q function, which gives the long-term value, or predicted average cumulative reward, of taking an action in the context of a particular user state. In one embodiment, the Q function estimate is implemented using a random forest regression model, a widely-used machine learning function approximation method. Other supervised learning regression methods (i.e. an algorithm that constructs an approximation of an unknown real-valued function based on a possibly noisy training data set), including multilayer neural networks, may be substituted without changing other components. Note that, in some implementations, other methods may not give the same learning/adaptation performance, or may require selection of tuning parameters to give good performance. The value learning module 102 performs one or more tasks. For instance, one task comprises executing the learned policy. The value learning module 102 receives a request from the controller module 88 to choose an action for the current user state. In one embodiment, the value learning module 102 uses a greedy action selection method. For instance, from the set of allowable actions (e.g., as determined by the constraints module 104), the action with the highest estimated Q value is chosen, with random selection in case of a tie. The following example pseudo code is illustrative of an example greedy action selection method:

```
Inputs:
  s (current user state)
  a_{L} (learned action)
Available Methods/Components:
  Q (current Q function estimate)
  Acts (allowable actions, as computed by
  the constraints module 104)
Outputs:
  a (selected action)
Method:
  A = Acts(s)
  qₘₐₓ = max {Q(s, a) for a in A}
  A’ = {a in A where Q(s,a) == qₘₐₓ}
  a = random (A’)
```

Though described using a greedy method, in some embodiments, other methods may be used, including Softmax methods, which make a weighted random choice from the set of allowable actions, where actions with higher estimated Q values have a greater probability of being chosen. In some embodiments, Thompson sampling methods use a Q function estimate that also gives some estimate of uncertainty or variance, and instead of comparing estimated Q values (as in the greedy method), randomly drawn Q values from the posterior distribution of the Q estimates for each action are compared. Additionally, in some embodiments, the value learning module 102 periodically (e.g. daily, though not limited as such) updates the Q function estimate based on observed states and rewards, as stored in the experience memory module 100. In one embodiment, the eHealth adaptive learning system uses a variant of a batch-fitted Q learning algorithm. An example of pseudocode for updating the Q function estimates may be as follows:

```
  Inputs:
    Q₀ (current Q function estimate)
  Available Methods/Components:
    Sample (draw a sample of transitions from experience memory)
    Fit (fit a random forest model to minimize root mean square
    error on a
training set)
    Acts (allowable actions, as computed by constraints module)
  Parameters:
    K (number of learning iterations, should be ≥ 1)
    Y (future reward discount factor)
    N (experience sample size)
    M (experience sample maximum age)
  Outputs:
    Q = (new Q function estimate)
  Method:
    Q = Q₀
    Repeat K times:
      T = Sample (M,N)
      X, Y = {x₁, x₂, ..., x_{N}}, {y₁, y₂, ...y_{N}} where:
        For (s, a, s’, r) in T:
          qₘₐₓ = max {Q(s’, a’) for a’ in Acts(s’)}
          x = (s, a)
          y= r + Y * qₘₐₓ
      Q = Fit (X, Y)
```

Note that the value function update may be asynchronous with regard to value function evaluation, and thus may be easily distributed to separate processes or servers, potentially improving the performance and scalability of implementations.

The constraints module 104 is responsible for enforcing requirements on intervention actions the eHealth adaptive learning system should take or should avoid taking. Constraints may be used for one or more reasons, including complying with explicitly stated user desires (e.g. no prompts after midnight), improving performance by avoiding actions with known poor behavior (e.g., as specified by domain experts), and/or complying with current or future safety, regulatory, or certification requirements. Note that (as per the latter point) the constraints module 104 enables the eHealth adaptive learning system to give strong guarantees that its behavior obeys safety and/or regulatory requirements, even when it is not possible to fully specify that behavior in advance, as it adapts to individual users. Constraints may also be updated if requirements change while minimizing changes to the rest of the system. In one embodiment, the constraints module 104 executes a constraint function specified by system operators. Given the current user state, the constraint function returns a subset of actions that are allowable at the current time. This subset of actions is used by the value learning module 102, as shown in the greedy action selection pseudocode and updating Q function estimates pseudocode described above.

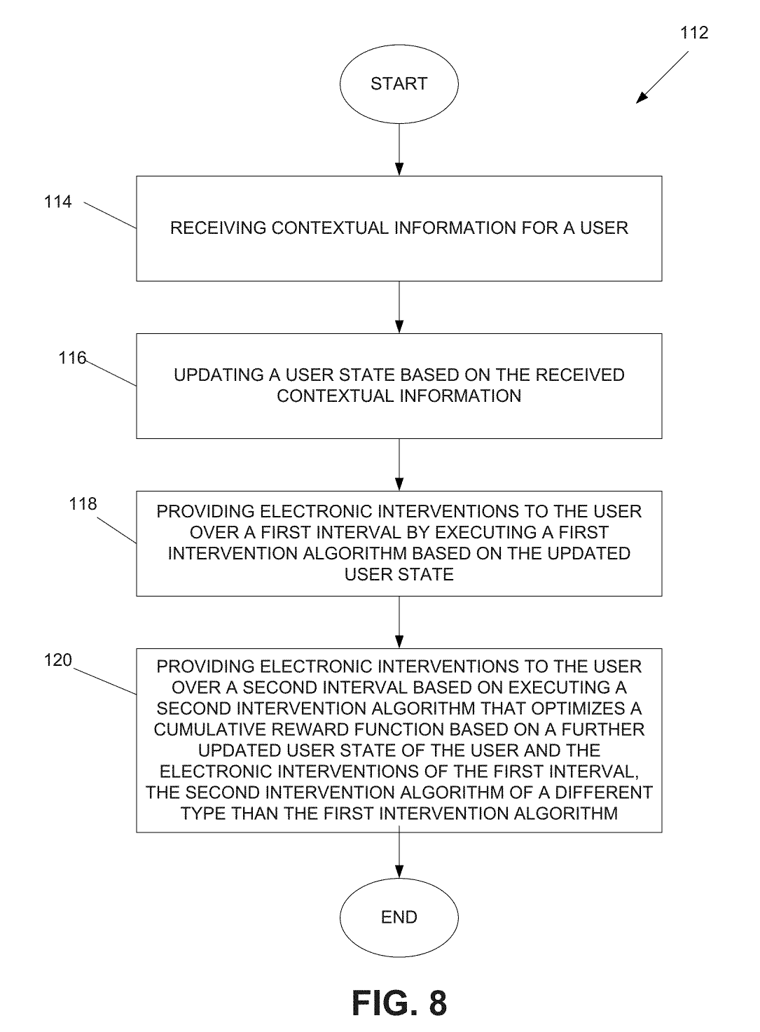

In view of the description above, it should be appreciated that one embodiment of an eHealth learning method 112 (e.g., implemented by the processing system 70 executing the application software 30B, FIGS. 4-5), depicted in FIG. 8 and encompassed between start and end designations, comprises receiving contextual information for a user (114); updating a user state based on the received contextual information (116); providing electronic interventions to the user over a first interval by executing a first intervention algorithm based on the updated user state (118); and providing electronic interventions to the user over a second interval based on executing a second intervention algorithm that maximizes a reward function based on a further updated user state of the user and the electronic interventions of the first interval, the second intervention algorithm of a different type than the first intervention algorithm (120).

Any process descriptions or blocks in the flow diagram described above should be understood as representing modules, segments, or portions of code which include one or more executable instructions for implementing specific logical functions or steps in the process, and alternate implementations are included within the scope of an embodiment of the present invention in which functions may be executed substantially concurrently, and/or additional logical functions or steps may be added, depending on the functionality involved, as would be understood by those reasonably skilled in the art of the present invention.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. For instance, as a fairly general-purpose learning and adaptation system, certain embodiments of an eHealth adaptive learning system may be broadly applicable. As one example, the eHealth adaptive learning system may be applied as a back-end component in a consumer-facing system that may be broadly characterized as a behavior change intervention to improve effectiveness, performance, adherence, or user satisfactions. Examples include products for personal health, products in which adherence is a concern, and suggesting care coordination tasks. The eHealth adaptive learning system may be provided as a service for third parties to use to supply enhanced health and lifestyle products. This approach may be desirable for applications where a client may wish to author their own highly-tailored health and wellness applications while still taking advantage of the learning and adaptation capabilities offered by the eHealth adaptive learning system. The eHealth adaptive learning system may be applied as part of a decision support system provided to behavioral medicine providers, such as substance abuse counsellors. The eHealth adaptive learning system may be similarly applied in educational settings for behavioral medicine providers. Note that various combinations of the disclosed embodiments may be used, and hence reference to an embodiment or one embodiment is not meant to exclude features from that embodiment from use with features from other embodiments. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical medium or solid-state medium supplied together with or as part of other hardware, but may also be distributed in other forms.

At least the following is claimed:

1. A system, comprising:
one or more storage devices comprising instructions; and
a processing system configured to execute the instructions to:
receive contextual information for a user;
update a user state based on the received contextual information;
provide electronic interventions to the user over a first interval by executing a first intervention algorithm based on the updated user state; and
provide electronic interventions to the user over a second interval based on executing a second intervention algorithm that maximizes a reward function based on a further updated user state of the user and the electronic interventions of the first interval, the second intervention algorithm of a different type than the first intervention algorithm.

2. The system of claim 1, wherein the processing system is configured to execute the instructions to provide the electronic interventions during a transition interval overlapping the first and second intervals, wherein during the transition interval, a probability of providing the electronic interventions of the first interval decreases while a probability of providing the electronic interventions of the second interval increases.

3. The system of claim 1, wherein the first intervention algorithm comprises one of random selected actions, rules, or a previously learned model that is trained on an experience or experiences of one or more other users, simulated users, or a combination of the one or more other users and the simulated users.

4. The system of claim 1, wherein the processing system is configured to execute the instructions to determine a reward or penalty based on a prior electronic intervention by executing a reward function that is personalized to a specific behavior change for the user.

5. The system of claim 4, wherein the reward function receives as input a user state prior to the last electronic intervention, the last electronic intervention, a current user state, a time of the last electronic intervention, and a current time.

6. The system of claim 1, wherein the processing system is configured to execute the instructions to record current and prior user states, prior electronic interventions, and prior rewards.

7. The system of claim 6, further comprising a data structure configured to store a time-indexed sequence of transitions, wherein each of the transitions comprises:
a user state immediately before an electronic intervention;
the electronic intervention;
a user state after one unit of time has elapsed; and
a received award.

8. The system of claim 1, wherein the processing system is configured to execute the instructions to execute the second intervention algorithm to maintain an estimate of a Q function, the Q function comprising a long term value or predicted average cumulative reward based on providing an electronic intervention in the context of a particular user state.

9. The system of claim 8, wherein the Q function is estimated based on a supervised learning method.

10. The system of claim 8, wherein the processing system is configured to execute the instructions to select one of the electronic interventions of the second interval among a plurality of possible electronic interventions based on a current user state and with a highest estimated Q value.

11. The system of claim 10, wherein the processing system is configured to execute the instructions to use random selection when more than one of the electronic interventions among the possible electronic interventions has the same estimated Q value.

12. The system of claim 8, wherein the processing system is configured to execute the instructions to select one of the electronic interventions of the second interval among a plurality of possible electronic interventions based on a weighted random choice, wherein electronic interventions with higher estimated Q values have a greater probability of being selected.

13. The system of claim 8, wherein the processing system is configured to execute the instructions to select one of the electronic interventions of the second interval among a plurality of possible electronic interventions based on an estimated Q value and an estimate of variance, wherein randomly drawn Q values from a posterior distribution of the Q estimates are compared.

14. The system of claim 8, wherein the processing system is configured to execute the instructions to repeatedly update the Q function based on updates to the user state and rewards.

15. The system of claim 1, wherein the processing system is configured to execute the instructions to provide the electronic interventions based on enforcement of one or more constraints, wherein the one or more constraints are based on one or any combination of user input, performance constraints, safety, regulation, or certification.

16. A computer-implemented method, comprising:
receiving contextual information for a user;
updating a user state based on the received contextual information;
providing electronic interventions to the user over a first interval by executing a first intervention algorithm based on the updated user state; and
providing electronic interventions to the user over a second interval based on executing a second intervention algorithm that maximizes a reward function based on a further updated user state of the user and the electronic interventions of the first interval, the second intervention algorithm of a different type than the first intervention algorithm.

17. The method of claim 16, further comprising providing the electronic interventions during a transition interval overlapping the first and second intervals, wherein during the transition interval, a probability of providing the electronic interventions of the first interval decreases while a probability of providing the electronic interventions of the second interval increases.

18. The method of claim 16, wherein the first intervention algorithm comprises one of random selected actions, rules, or a previously learned model that is trained on an experience or experiences of one or more other users, simulated users, or a combination of the one or more other users and the simulated users, further comprising:
determining a reward or penalty based on a prior electronic intervention by computing a reward function that is personalized to a specific behavior change for the user, wherein the computing of the reward function is based on receiving as input a user state prior to the last electronic intervention, the last electronic intervention, a current user state, a time of the last electronic intervention, and a current time;
wherein executing the second intervention algorithm comprises maintaining an estimate of a Q function, the Q function comprising a long term value or predicted average cumulative reward based on providing an electronic intervention in the context of a particular user state, wherein the estimate of the Q function is based on implementing a supervised learning method.

19. The method of claim 18, further comprising:
selecting one of the electronic interventions of the second interval among a plurality of possible electronic interventions based on a current user state and with a highest estimated Q value;
selecting one of the electronic interventions of the second interval among a plurality of possible electronic interventions based on one of a weighted random choice, wherein electronic interventions with higher estimated Q values have a greater probability of being selected or an estimated Q value and an estimate of variance, wherein randomly drawn Q values from a posterior distribution of the Q estimates are compared;
repeatedly updating the Q function based on updates to the user state and rewards; and
providing the electronic interventions based on enforcement of one or more constraints, wherein the one or more constraints are based on one or any combination of user input, performance constraints, safety, regulation, or certification.

20. A non-transitory, computer readable medium comprising instructions that, when executed by a processing system, causes the processing system to:
receive contextual information for a user;
update a user state based on the received contextual information;
provide electronic interventions to the user over a first interval by executing a first intervention algorithm based on the updated user state; and
provide electronic interventions to the user over a second interval based on executing a second intervention algorithm that maximizes a reward function based on a further updated user state of the user and the electronic interventions of the first interval, the second intervention algorithm of a different type than the first intervention algorithm.
